(19) United States
(12) Patent Application Publication
(10) Pub. No.: US 2023/0095592 A1
(43) Pub. Date: Mar. 30, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP); Kenji NAGAMIYA, Kanagawa (JP); Yasuyuki HOSONO, Kanagawa (JP); Nobuya KITAMURA, Kanagawa (JP)

(21) Appl. No.: 17/929,757

(22) Filed: Sep. 6, 2022

(30) Foreign Application Priority Data
Sep. 27, 2021 (JP) 2021-157271

(51) Int. Cl. G16H 10/20; H04L 51/02

(52) U.S. Cl. CPC G16H 10/20; H04L 51/02

(57) ABSTRACT

An information processing apparatus includes at least one processor. The processor acquires at least one of input information, which is information input to the information processing apparatus, or biological information of a user, asks a question about a physical condition of the user in a case in which it is determined that an abnormality occurs in the physical condition of the user based on at least one of the input information or the biological information, and presents a comment based on an answer to the question.

### CROSS-REFERENCE TO RELATED APPLICATIONS

The present application claims priority under 35 U.S.C. § 119 to Japanese Patent Application No. 2021-157271, filed on Sep. 27, 2021. The above application is hereby expressly incorporated by reference, in its entirety, into the present application.

### BACKGROUND

### 1. Technical Field

The disclosed technology relates to an information processing apparatus, an information processing method, and an information processing program.

### 2. Description of the Related Art

The following technologies are known as technologies of managing the health of a user or determining a symptom through a conversation with the user. For example, JP2018-033660A discloses a medical support system comprising a determination unit that determines a degree of a symptom of a subject in a case in which an abnormality of the subject is detected based on vital data of the subject, in which, in a case in which the degree of the symptom of the subject cannot be determined, a question to confirm the symptom of the subject is output.

JP2013-073509A discloses a communication system comprising a user communication terminal used by a user, a plurality of instructor communication terminals used to instruct the user, and a server that communicates with the user communication terminal, in which the server displays a question about a body and life of the user on a display unit of the user communication terminal, and transmits an answer to the question to any of the instructor communication terminals.

### SUMMARY

In recent years, an application that manages the health of the user by daily measuring biological information, such as a heartbeat, a blood pressure, and an electrocardiography of the user by using a wearable device, such as a smart watch, has been put into practical use. However, it is troublesome for the user to check the biological information diligently, and the user may not be aware of the abnormality of the biological information. In addition, even in a case in which the abnormality occurs in his/her physical condition, in a case in which the user does not perceive his/her symptom, the user may leave the symptom as it is without being aware of the abnormality of the physical condition, so that the symptom may deteriorate.

The disclosed technology has been made in view of the above points, and is to urge the user to be aware of the abnormality in a case in which it is presumed that the abnormality occurs in the physical condition of the user.

The disclosed technology relates to an information processing apparatus comprising at least one processor. The processor performs a process of acquiring at least one of input information, which is information input to the information processing apparatus, or biological information of a user, asking a question about a physical condition of the user in a case in which it is determined that an abnormality occurs in the physical condition of the user based on at least one of the input information or the biological information, and presenting a comment based on an answer to the question.

The input information may include a search keyword input to a search engine. In addition, the input information may include a content transmitted or sent to a specified or unspecified party. In addition, the input information may include a word uttered by the user in a conversation with a chatbot operated on the information processing apparatus.

The biological information may be a measurement value of an index indicating a health state of the user. In this case, the processor may determine that the abnormality occurs in the physical condition of the user in a case in which the measurement value is not within a predetermined range, or the processor may determine that the abnormality occurs in the physical condition of the user in a case in which a fluctuation amount of the measurement value is larger than a predetermined amount.

The comment may include a further question. The processor may include, in the comment, a content to which the biological information is added. The processor may include, in the comment, a content to which input information or biological information at a point in time before an acquisition point in time of the input information or the biological information that triggers asking the question is added. The processor may repeat a content of the answer in the comment. The processor may include, in the comment, behavior recommended to the user.

The disclosed technology relates to an information processing method executed by at least one processor provided in an information processing apparatus, the method comprising acquiring at least one of input information, which is information input to the information processing apparatus, or biological information of a user, asking a question about a physical condition of the user in a case in which it is determined that an abnormality occurs in the physical condition of the user based on at least one of the input information or the biological information, and presenting a comment based on an answer to the question.

The disclosed technology relates to an information processing program causing at least one processor provided in an information processing apparatus to execute a process comprising acquiring at least one of input information, which is information input to the information processing apparatus, or biological information of a user, asking a question about a physical condition of the user in a case in which it is determined that an abnormality occurs in the physical condition of the user based on at least one of the input information or the biological information, and presenting a comment based on an answer to the question.

According to the disclosed technology, in a case in which it is presumed that the abnormality occurs in the physical condition of the user, it is possible to urge the user to be aware of the abnormality.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a health management system according to an embodiment of the disclosed technology;

FIG. 2 is a diagram showing an example of a hardware configuration of an information processing apparatus;

FIG. 3 is a functional block diagram showing an example of a functional configuration of the information processing apparatus according to the embodiment of the disclosed technology;

FIG. 4 is a diagram showing an example of a conversation between a chatbot and a user;

FIG. 5 is a diagram showing another example of the conversation between the chatbot and the user;

FIG. 6 is a diagram showing still another example of the conversation between the chatbot and the user;

FIG. 7 is a diagram showing still another example of the conversation between the chatbot and the user; and

FIG. 8 is a flowchart showing an example of a flow of a process performed by executing a health management program according to the embodiment of the disclosed technology.

### DETAILED DESCRIPTION

In the following, an example of an embodiment of the disclosed technology will be described with reference to the drawings. It should be noted that the same or equivalent components and portions in the drawings are represented by the same reference numerals, and the overlapping description will be omitted.

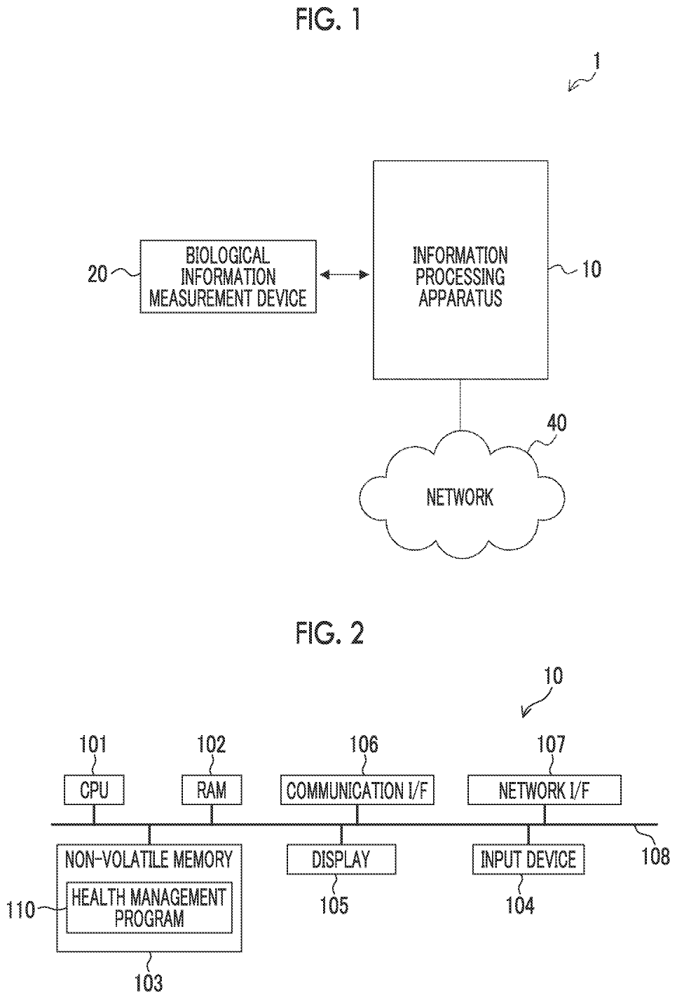

FIG. 1 is a diagram showing an example of a configuration of a health management system 1 according to an embodiment of the disclosed technology. The health management system 1 includes an information processing apparatus 10 and a biological information measurement device 20.

The information processing apparatus 10 is a communication terminal apparatus used by a user who is a management target subject by the health management system 1. The information processing apparatus 10 may be, for example, a desktop computer, a laptop, a tablet computer, or a smartphone. The information processing apparatus 10 is connected to a network 40 that can be used by the public, such as the Internet. The information processing apparatus 10 can access a web server (not shown) via the network 40, or can perform a web search by using a search engine.

The biological information measurement device 20 measures biological information of the user. The biological information may be a measurement value of an index indicating a health state of the user, for example, may be information indicating at least one of a body temperature, a heartbeat, electrocardiography, myoelectricity, a blood pressure, arterial oxygen saturation (SpO2), a blood glucose level, a lipid level, or the like. The biological information measurement device 20 may be a wearable device, such as a smart watch, comprising a sensor that measures these pieces of the biological information. The biological information measurement device 20 has a function of continuously measuring the biological information of the user in accordance with a predetermined time schedule and storing the biological information in an internal memory. The biological information measurement device 20 may measure the biological information for each predetermined period (for example, at intervals of 30 minutes). The biological information measurement device 20 is communicably connected to the information processing apparatus 10, and transmits the measured biological information to the information processing apparatus 10.

FIG. 2 is a diagram showing an example of a hardware configuration of the information processing apparatus 10. The information processing apparatus 10 includes a central processing unit (CPU) 101, a random access memory (RAM) 102, a non-volatile memory 103, an input device 104 including a keyboard, a mouse, a microphone, and the like, a display 105, a communication interface 106, and a network interface 107. The hardware is connected to a bus 108.

The display 105 may be a touch panel display. The communication interface 106 is an interface for the information processing apparatus 10 to perform communication with the biological information measurement device 20. A communication method may be any of wired or wireless. For wireless communication, for example, a method compliant with an existing wireless communication standard, such as Wi-Fi (registered trademark) and Bluetooth (registered trademark), can be applied. The network interface 107 is an interface for the information processing apparatus 10 to be connected to the network 40. A communication method may be any of wired or wireless. For wireless communication, for example, a method compliant with an existing wireless communication standard, such as Wi-Fi (registered trademark), can be applied.

The non-volatile memory 103 is a storage medium, such as a hard disk and a flash memory, and stores a health management program 110. The RAM 102 is a work memory for the CPU 101 to execute a process. The CPU 101 loads the health management program 110 stored in the non-volatile memory 103 into the RAM 102, and executes the process in accordance with the health management program 110. The CPU 101 is an example of a “processor” according to the disclosed technology.

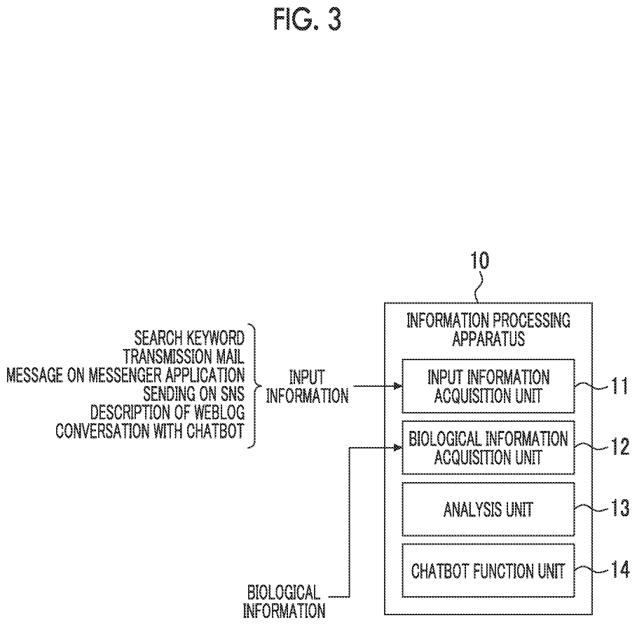

FIG. 3 is a functional block diagram showing an example of a functional configuration of the information processing apparatus 10. The information processing apparatus 10 includes an input information acquisition unit 11, a biological information acquisition unit 12, an analysis unit 13, and a chatbot function unit 14. By the CPU 101 executing the health management program 110, the CPU 101 functions as the input information acquisition unit 11, the biological information acquisition unit 12, the chatbot function unit 14, and the analysis unit 13.

The input information acquisition unit 11 acquires input information input to the information processing apparatus 10. The input information may include a word input to the information processing apparatus 10. The input of the word as the input information can be performed by using, for example, the keyboard or the microphone as the input device 104. The input information may be information input on various applications installed in the information processing apparatus 10.

For example, the input information may include a search keyword input to a search engine for searching for information on a network. In addition, the input information may include a word transmitted or sent to a specified or unspecified party, such as a transmission mail, a message on a messenger application, sending on social networking service (SNS), description of a weblog, or a chat conversation. In addition, the input information may include a word uttered by the user in a conversation with a chatbot operated on the information processing apparatus 10. The chatbot that is operated on the information processing apparatus 10 is realized by the chatbot function unit 14. The input information acquisition unit 11 may acquire a combination of any two or more of information described above as the input information.

The input information acquisition unit 11 acquires the input information by linking with various applications, such as the web browser, an e-mail application, a messenger application, and an SNS application installed in the information processing apparatus 10.

The biological information acquisition unit 12 acquires the biological information of the user measured by the biological information measurement device 20. The biological information acquisition unit 12 acquires the measurement value of the body temperature, the heartbeat, the electrocardiography, the myoelectricity, the blood pressure, the arterial oxygen saturation (SpO2), the blood glucose level, the lipid level, or the like as the biological information from the biological information measurement device 20 by the communication with the biological information measurement device 20. In a case in which the biological information is intermittently measured by the biological information measurement device 20, the biological information acquisition unit 12 may acquire the biological information each time the measurement is performed by the biological information measurement device 20. In a case in which the biological information is time-series data, such as the electrocardiographic waveform, the biological information acquisition unit 12 may acquire the time-series data over a predetermined period (for example, 1 minute) as the biological information in one measurement. In addition, in a case in which the time-series data, such as the electrocardiographic waveform, is always measured by the biological information measurement device 20, the biological information acquisition unit 12 may always acquire the time-series data. The biological information acquisition unit 12 stores the acquired biological information in the non-volatile memory 103.

The analysis unit 13 analyzes the input information acquired by the input information acquisition unit 11 and the biological information acquired by the biological information acquisition unit 12, and determines whether or not an abnormality occurs in a physical condition of the user based on the input information and the biological information. The analysis unit 13 may determine that the abnormality occurs in the physical condition of the user in a case in which, for example, a word, which is registered in advance as a word related to the physical condition, a symptom, health, feeling, emotion, or a disease name of the user, is included in the word as the input information.

For example, in a case in which the input information is the search keyword input to the search engine, in a case in which the word registered in advance, such as “fever”, “cough”, “headache”, “stomach ache”, “dizzy”, “cold”, “I cannot sleep”, “anxiety”, “I feel depressed”, “I feel listless”, “I get annoyed”, “influenza”, “novel coronavirus”, or “COVID-19” is included in the search keyword, the analysis unit 13 may determine that the abnormality occurs in the physical condition of the user. In addition, for example, in a case in which the input information is the message input on the messenger application, in a case in which the registered word described above is included in the message, the analysis unit 13 may determine that the abnormality occurs in the physical condition of the user. In addition, in a case in which the user requests the chatbot realized by the chatbot function unit 14 to present the biological information, the analysis unit 13 may determine that the abnormality occurs in the physical condition of the user.

In addition, in a case in which the measurement value of the body temperature, the heartbeat, the electrocardiography, the myoelectricity, the blood pressure, the arterial oxygen saturation (SpO2), the blood glucose level, the lipid level, or the like as the biological information is not within a predetermined range, the analysis unit 13 may determine that the abnormality occurs in the physical condition of the user. The predetermined range described above may be a predetermined fixed value, or may be a range determined based on the measurement value of the user in a most recent predetermined period. In addition, in a case in which a fluctuation amount of the measurement value as the biological information is larger than a predetermined amount, the analysis unit 13 may determine that the abnormality occurs in the physical condition of the user. The fluctuation amount described above may be, for example, a fluctuation amount from an average of the measurement values of the user in the most recent predetermined period (for example, one week).

The chatbot function unit 14 has a function of responding to a text or voice input by the user using the input device 104. The chatbot function unit 14 is constructed by using, for example, known natural language processing technique and machine learning. The user can request the chatbot realized by the chatbot function unit 14 to present the biological information (for example, the measurement value of the body temperature) through the text or the voice. In a case in which the user requests the presentation of the biological information, the chatbot function unit 14 outputs the biological information acquired by the biological information acquisition unit 12 as a response. In addition, the user can also request the chatbot to perform the web search by designating any search keyword. In a case in which the user requests to perform the web search, the chatbot function unit 14 performs the web search based on the designated search keyword and outputs the result as a response.

In a case in which the analysis unit 13 determines that the abnormality occurs in the physical condition of the user based on at least one of the input information or the biological information, the chatbot function unit 14 asks a question about the physical condition of the user. In addition, the chatbot function unit 14 presents a comment based on an answer from the user to the question described above.

### Conversation Example 1

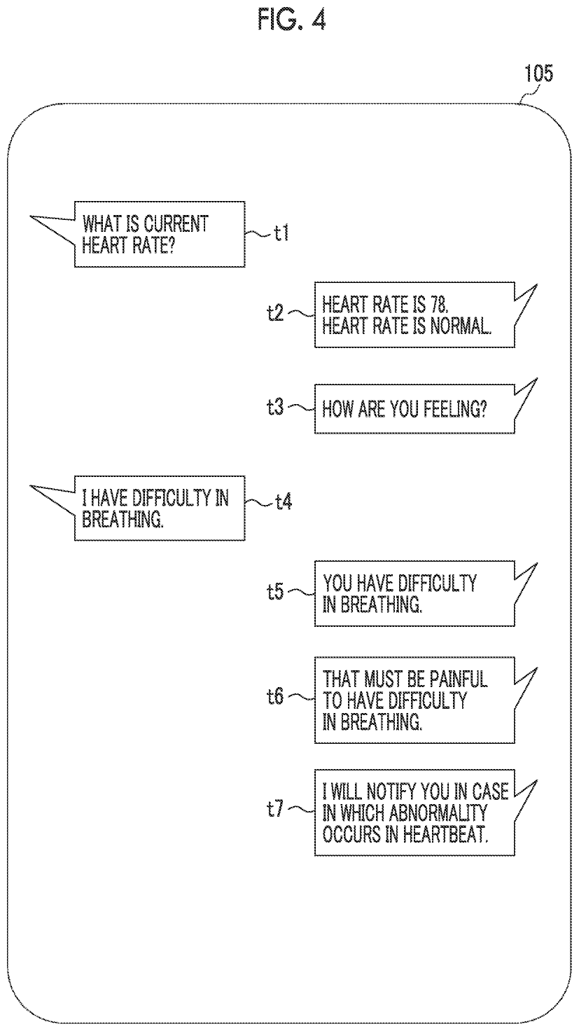

FIG. 4 is a diagram showing an example of the conversation between the chatbot realized by the chatbot function unit 14 and the user. The example shown in FIG. 4 shows a case in which a word uttered by the user in the conversation with the chatbot is acquired as the input information, and the analysis unit 13 determines that the abnormality occurs in the physical condition of the user based on the input information. A text indicating a content of the conversation between the chatbot and the user is displayed on a display screen of the display 105.

In the example shown in FIG. 4, the user inputs “What is the current heart rate?” to request the chatbot to present the heart rate as the biological information (conversation t1). The content of the conversation is acquired as the input information. On the other hand, the chatbot responds “The heart rate is 78” based on the biological information acquired by the biological information acquisition unit 12 (conversation t2). On the other hand, the analysis unit 13 determines that the abnormality occurs in the physical condition of the user based on the request of the user for the presentation of the biological information. In response to this, the chatbot asks a question about the physical condition of the user, saying, “How are you feeling?” (conversation t3). In response to this question, the user answers the symptom that he/she is aware of, saying, “I have difficulty in breathing” (conversation t4). In response to this answer, the chatbot presents a comment that repeats the content of the answer of the user, saying, “You have difficulty in breathing, don't you?” (conversation t5). Further, the chatbot presents the comment that infers the physical or mental distress of the user due to a subjective symptom of the user, saying, “That must be painful to have difficulty in breathing” (conversation t6). In addition, the chatbot presents a comment that follow-up will be performed, saying, “I will notify you in a case in which the abnormality occurs in the heartbeat” (conversation t7).

In this way, in a case in which it is suspected that the abnormality occurs in the physical condition of the user, the chatbot can ask the user the question about the physical condition of the user (conversation t3) to urge the user to be aware of the abnormality in the physical condition. In addition, by presenting the comment that repeats the content of the answer from the user (conversation t5), or presenting the comment that infers the distress of the user (conversation t6), the chatbot can show the understanding of the symptom of the user.

### Conversation Example 2

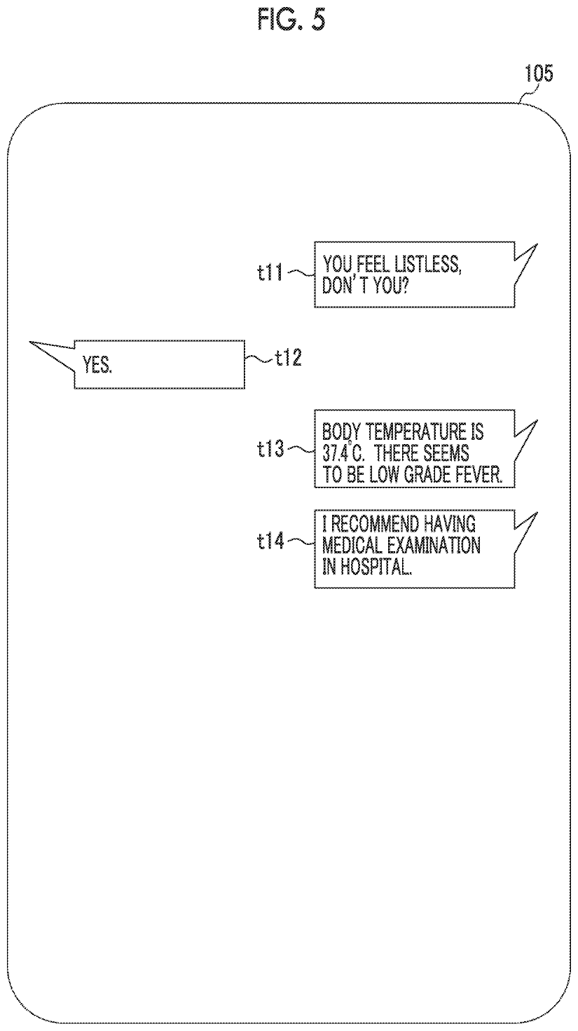

FIG. 5 is a diagram showing another example of the conversation between the chatbot realized by the chatbot function unit 14 and the user. The example shown in FIG. 5 shows a case in which the word of “I feel listless” sent from the user on the messenger application installed in the information processing apparatus 10 is acquired as the input information, and the analysis unit 13 determines that the abnormality occurs in the physical condition of the user based on the input information.

The chatbot asks a question about the physical condition of the user, saying, “You feel listless, don't you?” based on the word of “I feel listless” sent from the user (conversation t11). By asking this question, it is confirmed that a person who utters the word of “I feel listless” is the user, and the user is urged to be aware of the abnormality in the physical condition. The user answers “yes” to this question (conversation t12). In response to this answer, the chatbot presents a comment to which the body temperature as the biological information acquired by the biological information acquisition unit 12 is added, saying, “The body temperature is 37.4° C. There seems to be a low grade fever” (conversation t13). In addition, the chatbot presents a comment indicating behavior recommended to the user, saying, “I recommend having medical examination in a hospital” (conversation t14).

As in this example, the chatbot responds to the word input on other applications, in addition to the word uttered by the user towards the chatbot. That is, even in a case in which it is determined that the abnormality occurs in the physical condition of the user based on the input information input on the other applications, the chatbot asks the question about the physical condition of the user based on the input information (conversation t11). As a result, it is possible to urge the user to be aware of the abnormality in physical condition. In addition, in a case in which the chatbot presents the comment to which the biological information is added (conversation t13), it is possible to present information supporting the abnormality in the physical condition. In addition, by including the behavior recommended by the chatbot to the user in the comment (conversation t14), the user can be urged to execute the behavior.

### Conversation Example 3

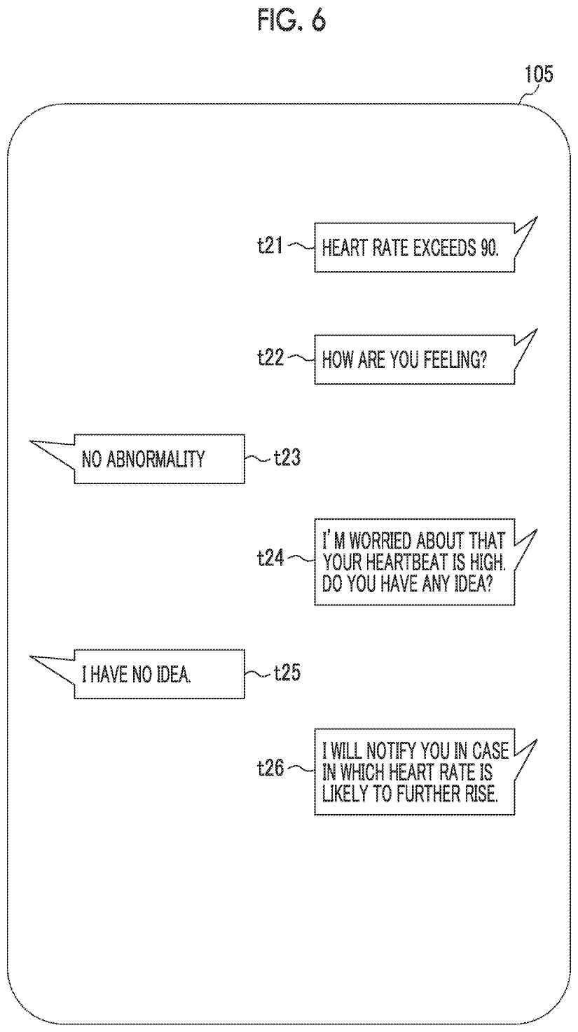

FIG. 6 is a diagram showing still another example of the conversation between the chatbot realized by the chatbot function unit 14 and the user. The example shown in FIG. 6 shows a case in which the analysis unit 13 determines that the abnormality occurs in the physical condition of the user based on the biological information (heart rate).

The chatbot notifies the user that the biological information indicates the abnormality, saying, “The heart rate exceeds 90” in response to the measurement value of the heart rate as the biological information indicating the abnormality (conversation t21). Subsequently, the chatbot asks a question about the physical condition of the user, saying, “How are you feeling?”, and urges the user to be aware of the abnormality in the physical condition (conversation t22). The user answers “no abnormality” to this question (conversation t23). In response to this answer, the chatbot presents a comment to which a content including the biological information (heart rate) and a further question is added, saying, “I'm worried about that your heartbeat is high. Do you have any idea?” (conversation t24). The user answers “I have no idea” to this question (conversation t25). In response to this answer, the chatbot presents a comment to the effect that monitoring the biological information will be continued, saying, “I will notify you in a case in which the heart rate is likely to further rise” (conversation t26).

As in this example, in a case in which the analysis unit 13 determines that the abnormality occurs in the physical condition of the user based on the biological information, the chatbot notifies the user of the abnormality. As a result, it is possible to notify the user that there is a possibility that the abnormality occurs in the physical condition of the user. In addition, in a case in which it is suspected that the abnormality occurs in the physical condition of the user, the chatbot can ask the user the question about the physical condition of the user (conversation t22) to urge the user to be aware of the abnormality in the physical condition. In addition, the chatbot can urge the user to perceive his/her physical condition by presenting the comment including the further question in accordance with the answer from the user to the question (conversation t24).

### Conversation Example 4

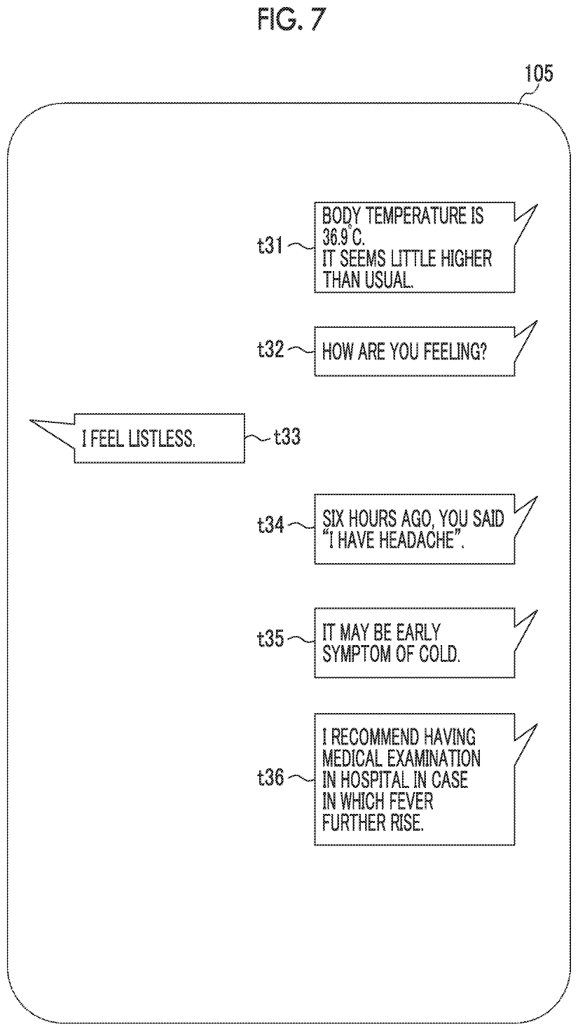

FIG. 7 is a diagram showing still another example of the conversation between the chatbot realized by the chatbot function unit 14 and the user. The example shown in FIG. 7 shows a case in which the analysis unit 13 determines that the abnormality occurs in the physical condition of the user based on the biological information (body temperature).

In accordance with the fact that the measurement value of the body temperature as the biological information is higher than the normal time, the chatbot notifies the user that the biological information is different from the normal time, saying, “The body temperature is 36.9° C. It seems a little higher than usual” (conversation t31). Subsequently, the chatbot asks a question about the physical condition of the user, saying, “How are you feeling?”, and urges the user to be aware of the abnormality in the physical condition (conversation t32). The user answers “I feel listless” to this question (conversation t33). In response to this answer, the chatbot presents a comment to which a content including the word of “I have a headache”, which is the past input information, is added, saying, “Six hours ago, you said “I have a headache”” (conversation t34). It should be noted that the past input information is the input information at a point in time before an acquisition point in time of the input information or the biological information that triggers the question about the physical condition of the user. In this example, it is assumed that the word of “I have a headache” uttered by the user six hours that is the point in time before the acquisition point in time of the body temperature indicating 36.9° C. is acquired as the input information. It should be noted that the past input information and the biological information are stored in the non-volatile memory 103. The chatbot presents a comment including past input information stored in the non-volatile memory 103. A storage period of the input information and the biological information can be set to any period.

Subsequently, the chatbot presents a comment indicating a cause of the abnormality in the physical condition presumed from the biological information, the answer of the user to the question and the past input information, saying, “It may be an early symptom of a cold” (conversation t35). In addition, the chatbot presents a comment indicating behavior recommended to the user, saying, “I recommend having medical examination in a hospital in a case in which the fever further rise” (conversation t36).

In a case in which it is suspected that the abnormality occurs in the physical condition of the user, the chatbot can ask the user the question about the physical condition of the user (conversation t32) to urge the user to be aware of the abnormality in the physical condition. In addition, the chatbot can urge the user to perceive his/her symptom by presenting the comment to which the content including the past input information is added in accordance with the answer from the user to the question (conversation t34). It should be noted that, in the example shown in FIG. 7, the case of presenting the comment to which the content including the past input information is added has been described, but a comment including a content including the past biological information may be presented.

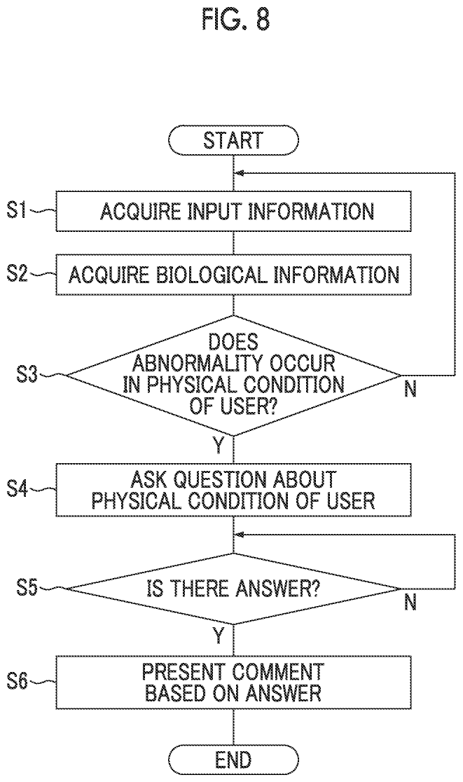

FIG. 8 is a flowchart showing an example of a flow of a process performed by the CPU 101 executing the health management program 110. The health management program 110 is executed, for example, in a case in which the user gives an instruction to start the process by operating the input device 104.

In step S1, the CPU 101 functions as the input information acquisition unit 11 and acquires the input information which is information input to the information processing apparatus 10.

In step S2, the CPU 101 functions as the biological information acquisition unit 12 and acquires the biological information from the biological information measurement device 20.

In step S3, the CPU 101 functions as the analysis unit 13 and determines whether or not the abnormality occurs in the physical condition of the user based on the input information acquired in step S1 and the biological information acquired in step S2. In a case in which it is determined that the abnormality occurs in the physical condition of the user, the process proceeds to step S4, and in a case in which it is determined that the abnormality does not occur in the physical condition of the user, the process returns to step S1.

In step S4, the CPU 101 functions as the chatbot function unit 14 and asks the question about the physical condition of the user. In step S5, the CPU 101 determines whether or not the answer from the user to the question is received.

In a case in which the answer from the user to the question is received, the CPU 101 functions as the chatbot function unit 14 and presents the comment based on the answer from the user received in step S5. This comment may be a repeat of the content of the answer from the user. In addition, this comment may also include a further question. For example, in a case in which the abnormality is confirmed in the biological information and the answer from the user to the question indicates that the abnormality does not occur in the physical condition of the user, the further question may be asked to ask the cause of the abnormality in the biological information. In addition, the comment may include a content to which the current biological information is added. In addition, the comment may include a content to which the past input information or the past biological information is added. In addition, the comment may also include the behavior recommended to the user.

As described above, the information processing apparatus 10 according to the embodiment of the disclosed technology acquires at least one of input information, which is information input to the information processing apparatus, or biological information of a user, asks a question about a physical condition of the user in a case in which it is determined that an abnormality occurs in the physical condition of the user based on at least one of the input information or the biological information, and presents a comment based on an answer to the question. With the information processing apparatus 10 according to the embodiment, in a case in which it is presumed that the abnormality occurs in the physical condition of the user, it is possible to urge the user to be aware of the abnormality. It should be noted that, in the present embodiment, the case has been described in which the information processing apparatus 10 acquires both the input information and the biological information, but a configuration may be adopted in which one of the input information or the biological information may is acquired.

In the embodiment described above, for example, as a hardware structure of processing units that execute various processes, such as the input information acquisition unit 11, the biological information acquisition unit 12, the analysis unit 13, and the chatbot function unit 14, the following various processors can be used. The various processors include, for example, a programmable logic device (PLD), such as an FPGA, which is a processor of which a circuit configuration can be changed after manufacture, and a dedicated electrical circuit, such as an application specific integrated circuit (ASIC), which is a processor having a dedicated circuit configuration designed to execute a specific process, in addition to the CPU and the GPU which are general-purpose processors that execute software (program) to function as various processing units as described above.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. A second example thereof is a form in which a processor that realizes all of the functions of a system including the plurality of processing units with one integrated circuit (IC) chip is used, as represented by a system on chip (SoC). In this way, various processing units are composed of one or more of the various processors as the hardware structure.

Further, more specifically, an electric circuit (circuitry) obtained by combining circuit elements, such as semiconductor elements, can be used as the hardware structure of the various processors.

In addition, in the embodiment described above, the aspect has been described in which the health management program 110 is stored (installed) in advance in the non-volatile memory 103, but the disclosed technology is not limited to this. The health management program 110 may be recorded on a recording medium, such as a compact disc read only memory (CD-ROM), a digital versatile disc read only memory (DVD-ROM), or a universal serial bus (USB) memory, and then provided. In addition, the health management program 110 may be downloaded from an external device via a network.

What is claimed is:

1. An information processing apparatus comprising:
at least one processor,
wherein the processor performs a process of
acquiring at least one of input information, which is information input to the information processing apparatus, or biological information of a user,
asking a question about a physical condition of the user in a case in which it is determined that an abnormality occurs in the physical condition of the user based on at least one of the input information or the biological information, and
presenting a comment based on an answer to the question.

2. The information processing apparatus according to claim 1,
wherein the input information includes a search keyword input to a search engine.

3. The information processing apparatus according to claim 1,
wherein the input information includes a content transmitted or sent to a specified or unspecified party.

4. The information processing apparatus according to claim 1,
wherein the input information includes a word uttered by the user in a conversation with a chatbot operated on the information processing apparatus.

5. The information processing apparatus according to claim 1,
wherein the biological information is a measurement value of an index indicating a health state of the user, and
the processor determines that the abnormality occurs in the physical condition of the user in a case in which the measurement value is not within a predetermined range.

6. The information processing apparatus according to claim 1,
wherein the biological information is a measurement value of an index indicating a health state of the user, and
the processor determines that the abnormality occurs in the physical condition of the user in a case in which a fluctuation amount of the measurement value is larger than a predetermined amount.

7. The information processing apparatus according to claim 1,
wherein the comment includes a further question.

8. The information processing apparatus according to claim 1,
wherein the processor includes, in the comment, a content to which the biological information is added.

9. The information processing apparatus according to claim 1,
wherein the processor includes, in the comment, a content to which input information or biological information at a point in time before an acquisition point in time of the input information or the biological information that triggers asking the question is added.

10. The information processing apparatus according to claim 1,
wherein the processor repeats a content of the answer in the comment.

11. The information processing apparatus according to claim 1,
wherein the processor includes, in the comment, behavior recommended to the user.

12. An information processing method executed by at least one processor provided in an information processing apparatus, the method comprising:
acquiring at least one of input information, which is information input to the information processing apparatus, or biological information of a user;
asking a question about a physical condition of the user in a case in which it is determined that an abnormality occurs in the physical condition of the user based on at least one of the input information or the biological information; and
presenting a comment based on an answer to the question.

13. A non-transitory computer-readable storage medium storing an information processing program causing at least one processor provided in an information processing apparatus to execute a process comprising:
acquiring at least one of input information, which is information input to the information processing apparatus, or biological information of a user;
asking a question about a physical condition of the user in a case in which it is determined that an abnormality occurs in the physical condition of the user based on at least one of the input information or the biological information; and
presenting a comment based on an answer to the question.
